(19) United States
(12) Patent Application Publication
(10) Pub. No.: US 2017/0164853 A1
(43) Pub. Date: Jun. 15, 2017

(54) METHOD FOR MANUFACTURING METAL ELECTRODE

(71) Applicant: ELECTRONICS AND TELECOMMUNICATIONS RESEARCH INSTITUTE, Daejeon (KR)

(72) Inventors: Yong Hee KIM, Daejeon (KR); Sang-Don JUNG, Daejeon (KR); Myung Ae CHUNG, Seoul (KR)

(21) Appl. No.: 15/220,321

(22) Filed: Jul. 26, 2016

(30) Foreign Application Priority Data
Dec. 15, 2015 (KR) 10-2015-0179385

(51) Int. Cl. A61B 5/04

(52) U.S. Cl. CPC A61B 5/04001; A61B 2562/125

(57) ABSTRACT

A method for manufacturing a metal electrode includes forming a resist pattern of which upper portion has a wider width than a lower portion thereof, forming an insulating layer for molding on the resist pattern, removing the resist pattern, thereby forming a mold, and forming, in the mold, a metal electrode including an alloy of a first metal and a second metal.

### CROSS-REFERENCE TO RELATED APPLICATION

The present application claims priority to Korean patent application number 10-2015-0179385 filed on Dec. 15, 2015, the entire disclosure of which is incorporated herein in its entirety by reference.

### BACKGROUND

1. Field

An aspect of the present disclosure relates to a technique for manufacturing technique a metal structure, and more particularly, to a method for controlling the size of a protruding metal electrode.

2. Description of the Related Art

When a neural signal is recorded, the distance between a nerve cell and a neural electrode has influence on the intensity of the neural signal. Also, when an electrical stimulation is applied to a nerve tissue, the distance between the neural tissue and a neural electrode has influence on the safety of the electrical stimulation. At this time, as the distance between the nerve cell or nerve tissue and the neural electrode becomes closer, it is more advantageous to record the neural signal and to apply the electrical stimulation. In typical planar electrode structures, there is a limitation in decreasing the distance between the nerve tissue and the neural electrode. On the other hand, in three-dimensional electrode structures having a hemispherical shape, a pyramid shape, a mushroom shape, a pin shape, and the like, it is advantageous to decrease the distance between the neural electrode and the nerve tissue.

When a neural signal is recorded, a high signal-to-noise ratio is required to perform an accurate signal analysis. Here, the neural signal has a unique value, and hence noise is reduced, thereby increasing the signal-to-noise ratio. The noise is in proportion to square root of impedance and in inverse proportion to surface area. Therefore, in order to reduce the noise, the impedance should be decreased or the surface area should be increased. In order to increase the surface area of an electrode, the electrode may be implemented using a nanoparticle, a nanowire, a nanorod, a nanoporous structure, etc.

A neural electrode of which surface is reformed to have a nanostructure has a large charge storage capacity. When the same current is applied to the neural electrode, the polarization of the neural electrode is smaller than that of the planar electrode. Hence, the neural electrode has a relatively large charge injection limit, and can safely stimulate nerve tissues without water decomposition.

Particularly, a micro protruding neural electrode having a micro size of 1 μm or less can measure a signal in a cell body, and hence it is advantageous to perform an accurate neural analysis. However, as the size of the neural electrode decreases, the contact area of the neural electrode with cells decreases, and hence it is disadvantageous in terms of signal measurement productivity.

### SUMMARY

Embodiments provide a method for manufacturing a protruding metal electrode having an increased surface area, and controlling the size of the protruding metal electrode.

According to an aspect of the present disclosure, there is provided a method for manufacturing a protruding metal electrode, the method including: forming a resist pattern of which upper portion has a wider width than a lower portion thereof; forming an insulating layer for molding on the resist pattern; removing the resist pattern, thereby forming a mold; and forming, in the mold, a metal electrode including an alloy of a first metal and a second metal.

### BRIEF DESCRIPTION OF THE DRAWINGS

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey the scope of the example embodiments to those skilled in the art.

In the drawing figures, dimensions may be exaggerated for clarity of illustration. It will be understood that when an element is referred to as being “between” two elements, it can be the only element between the two elements, or one or more intervening elements may also be present. Like reference numerals refer to like elements throughout.

FIGS. 1A to 1I are sectional views illustrating a method for manufacturing a metal electrode according to an embodiment of the present disclosure.

FIGS. 2A and 2B are electron microscope photographs obtained by photographing a protruding nanoporous metal electrode according to the embodiment of the present disclosure.

FIG. 3 illustrate photographs obtained by photographing, for every time zone, the protruding metal electrode formed using electron-co-deposition according to the embodiment of the present disclosure.

FIG. 4 is a conceptual view of an electrode array for an external nerve cell interface, to which the protruding metal electrode is applied according to the embodiment of the present disclosure.

### DETAILED DESCRIPTION

Hereinafter, exemplary embodiments of the present disclosure will be described. In the drawings, the thicknesses and the intervals of elements are exaggerated for convenience of illustration, and may be exaggerated compared to an actual physical thickness. In describing the present disclosure, a publicly known configuration irrelevant to the principal point of the present disclosure may be omitted. It should note that in giving reference numerals to elements of each drawing, like reference numerals refer to like elements even though like elements are shown in different drawings.

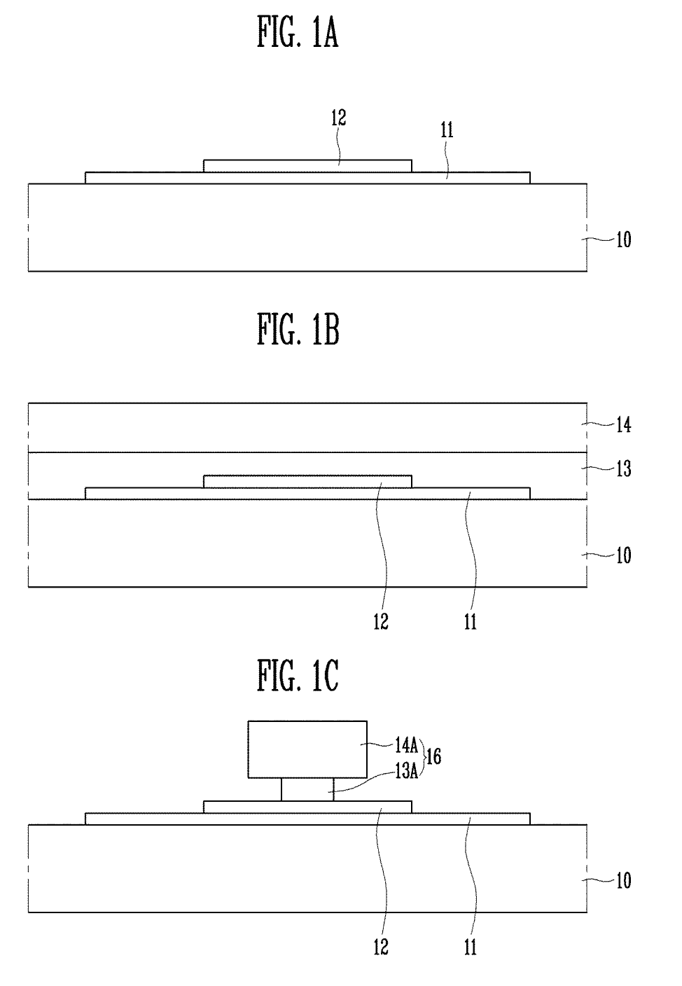

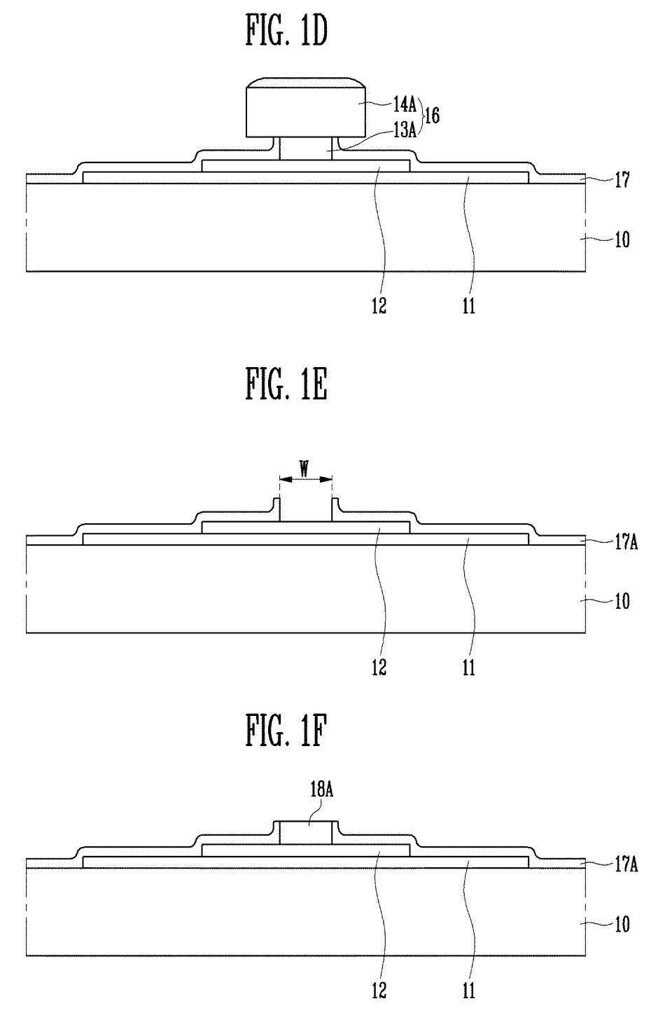

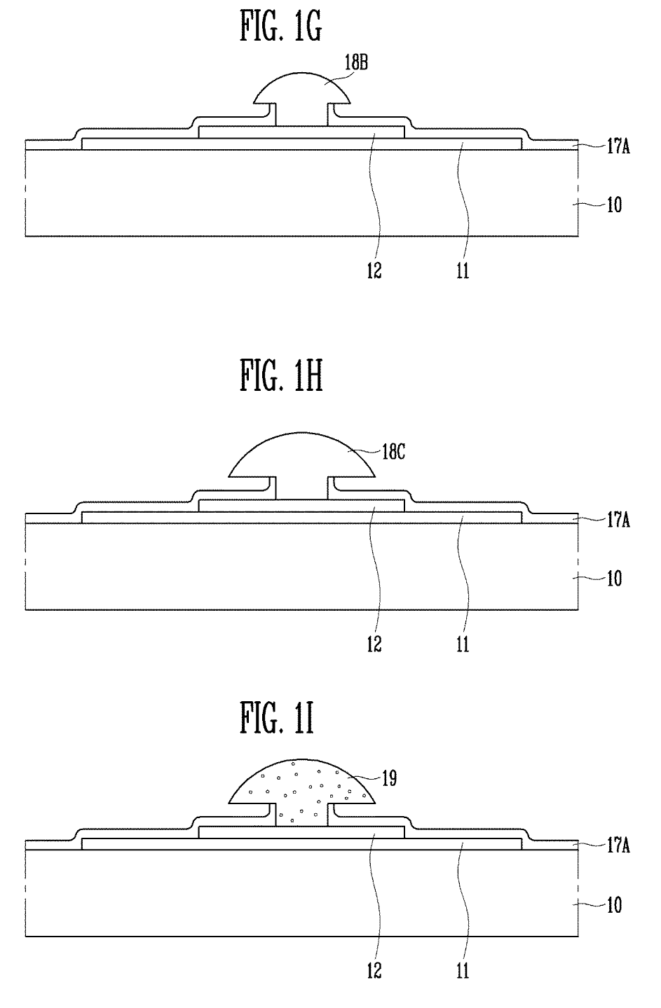

FIGS. 1A to 1I are sectional views illustrating a method for manufacturing a metal electrode according to an embodiment of the present disclosure.

Referring to FIG. 1A, a conductive pattern 11 and 12 is formed on a substrate 10. Here the conductive pattern 11 and 12 may be a metal thin film having a specific pattern. A first conductive pattern 11 may be a conductive pattern for wiring, and may include indium tin oxide (ITO). A second conductive pattern 12 may be a conductive pattern for electrodes.

Referring to FIG. 1B, a resist layer 13 and 14 is formed on the substrate 10 on which the conductive pattern 11 and 12 is formed. Here, the resist layer 13 and 14 may be formed in multiple layers, and may be formed by stacking various kinds of resist layers. For example, a lift-off resist 13 is formed using spin coating, and a negative photoresist 14 is then formed, thereby forming a double-layered resist structure. Here, the lift-off resist 13 may be formed of a material not sensitive to ultraviolet light. The lift-off resist 13 may be formed of a material not influenced by a solvent of the negative photoresist 14. The lift-off resist 13 may be a negative type resist. The negative photoresist 14 may include a polymer sensitive to ultraviolet light.

Referring to FIG. 1C, a resist pattern 16 is formed using a lithography process. First, there is performed an exposure process of irradiating ultraviolet light onto the negative photoresist 14 using a photomask having a desired electrode pattern formed therein and an exposure device having a photomask alignment function. A region exposed to the ultraviolet light in the negative photoresist 14 is crosslinked and cured, and becomes a thermally/mechanically stable state not subjected to glass transition. Subsequently, there is performed a development process of removing a region not exposed to the ultraviolet light in the negative photoresist 14 using a developer, thereby forming a negative photoresist pattern 14A. In addition, the non-exposure region of the negative photoresist 14 is removed, and simultaneously, a portion of the exposed lift-off resist 13 is removed using the same developer, thereby forming a lift-off resist pattern 13A. Accordingly, the resist pattern 16 is formed such that its upper portion has a wider width than its lower portion. The width of the lift-off resist pattern 13A may be adjusted depending on a development time. For example, the resist pattern 16 is formed as a resist pattern having an undercut or overhang structure such as a T shape or a gamma shape.

Referring to FIG. 1D, an insulating layer 17 for molding is formed over the resist pattern 16. For example, an intermediate resultant structure including the resist pattern 16 is cleaned and dried using water, and the insulating layer 17 for molding is coated over the entire surface of the intermediate resultant structure. In this case, a region in which the insulating layer 17 for molding is formed may be adjusted depending on a structure of the resist pattern 16. When the lift-off resist pattern 13A has a narrower width than the negative photoresist pattern 14A, the insulating layer 17 for molding may be formed on a sidewall of the lift-off resist pattern 13A and an upper surface of the negative photoresist pattern 14A. Also, the insulating layer 17 for molding may include SiO₂, Si₃N₄, Al₂O₃, etc.

In a sputtering process, the kinetic energy of plasma is converted into thermal energy to heat a sample. Therefore, the insulating layer 17 for molding is coated while the sample is being cooled using cooling water, etc. If the temperature of the sample is higher than a glass transition temperature of the lift-off resist pattern 13A, e.g., 180 to 190° C., the shape of the lift-off resist pattern 13A may be structurally changed. When the undercut length of the lift-off resist pattern 13A is short or when the amount of oxygen supplied in the sputtering process of forming the insulating layer 17 for molding using SiO₂ is sufficient, plasma particles reach the surface of the lift-off resist pattern 13A and then is easily diffused on the surface of the lift-off resist pattern 13A. Thus, the insulating layer 17 for molding is formed to surround the sidewall of the lift-off resist pattern 13A. For reference, although not shown in this figure, the insulating layer 17 for molding may also be thinly formed on a sidewall of the negative photoresist pattern 14A.

Referring to FIG. 1E, a mold 17A having a micro well structure is formed by removing the resist pattern 16. For example, the resist pattern 16 and the insulating layer 17 for molding, formed on the resist pattern 16 may be removed through a lift-off process using N-methylpyrrolidone.

If the resist pattern 16 is removed, an empty space is created inside the insulating layer 17 for molding, formed on the sidewall of the lift-off resist pattern 13A. Here, since the empty space has a width W of a few micrometers, the empty space is referred to as a micro well. The detailed structure of the micro well is determined by the undercut length of the resist pattern 16, the thickness of the lift-off resist pattern 13A, and the thickness of the sputtered insulating layer 17 for molding, and the depth of the micro well may have a value greater than the thickness of the insulating layer 17 for molding.

Referring to FIGS. IF to 1H, a metal electrode 18A to 18C including an alloy of a first metal and a second metal is formed in the mold 17A having the structure of the micro well. For example, an alloy layer is filled in the micro well using electro-co-deposition. In this case, the size of the metal electrode 18A to 18C may be controlled depending on a time required to deposit the alloy layer. Since the alloy is filled in the micro well in an early stage of the deposition, the metal electrode 18A is formed into a rod structure as shown in FIG. 1F. If the deposition is continuously performed, a protruding portion is formed as shown in FIG. 1G, thereby forming a protrusion-shaped metal electrode 18B. If the deposition is continuously performed, the size of a protruding portion of a metal electrode 18C be increased as shown in FIG. 1H, and the diameter of the protruding portion may be increased to be three times greater than the width W of the micro well, i.e., the diameter of the rod structure. For example, a rod structure corresponding to a stipe of a mushroom structure is formed in an early stage of a deposition process, and, if the deposition process is continuously performed, a mushroom head having a wider width than the stipe is formed. In addition, as the time required to perform the deposition process is increased, the size of the mushroom head is increased, and the diameter of the mushroom head may be increased to be three times greater than the width W of the micro well, i.e., the diameter of the stipe.

The first metal and the second metal, which are included in the metal electrode 18A to 18C, may be selected depending on a solubility with respect to a specific etchant. A metal dissolved in the specific etchant may be selected as the first metal, and a metal not dissolved in the specific etchant may be selected as the second metal. As an example, silver (Ag) dissolved in a nitric acid may be selected as the first metal, and gold (Au) not dissolved in the nitric acid may be selected as the second metal, thereby forming an Ag—Au alloy layer 18A to 18C. As another example, Au may be selected as the first metal, and platinum not dissolved in a gold etchant may be selected as the second metal, thereby forming an Au—Pt alloy layer 18A to 18C.

Referring to FIG. 1I, the first metal included in the metal electrode 18C is selectively removed using an etchant, thereby forming a protruding nanoporous metal electrode 19. As an example, if the first metal (Ag) of the metal electrode 18C including an Ag—Au alloy is removed using a silver etchant such as a nitric acid, a protruding nanoporous Au electrode is finally obtained. As another example, if the first metal (Au) of the metal electrode 18C including an Au—Pt alloy is removed using a gold etchant, a protruding nanoporous Pt electrode is finally obtained.

Here, the protruding nanoporous metal electrode 19 may be used as a neural electrode such as an electrode for extracellular neural signal detecting or an electrode for neural electrical stimulation. The protruding nanoporous metal electrode 19 may be a neural electrode included in an electrode array for an external nerve cell interface.

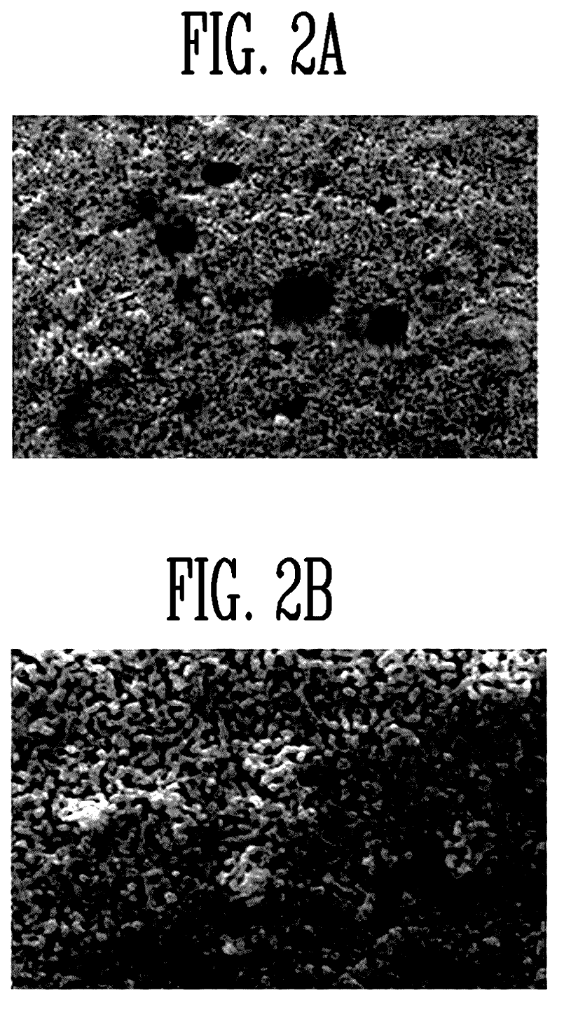

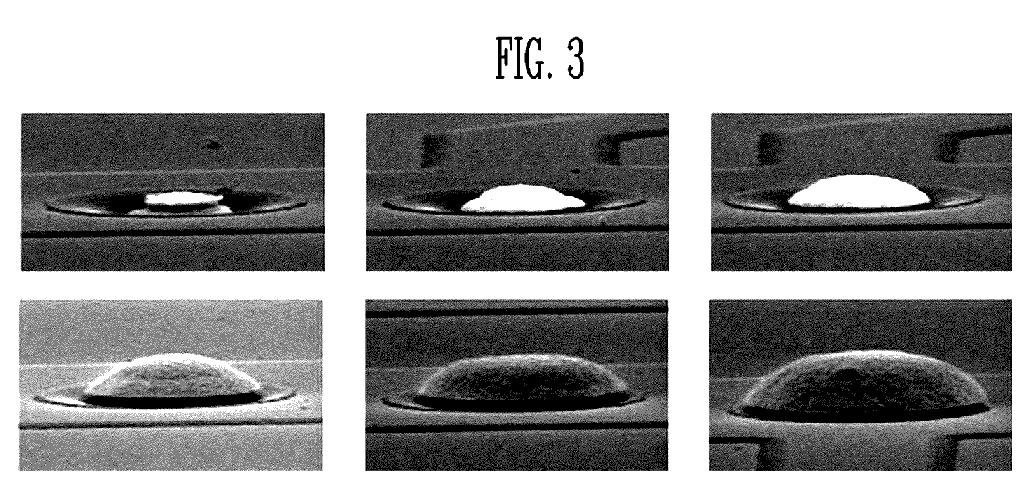

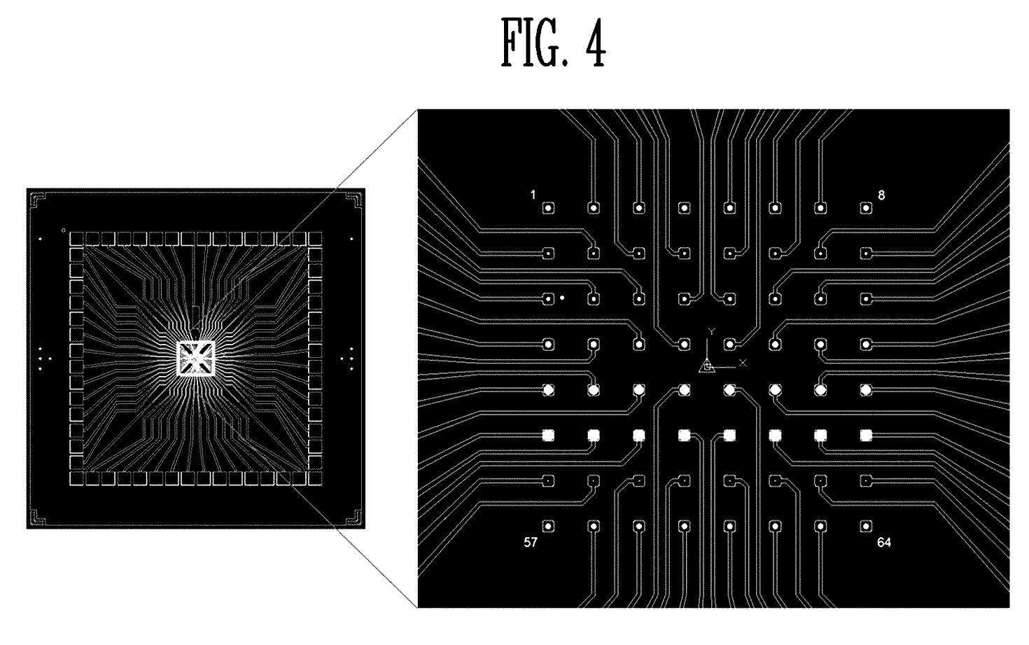

FIGS. 2A and 2B are electron microscope photographs obtained by photographing the protruding nanoporous metal electrode according to the embodiment of the present disclosure. FIG. 2A is a photograph obtained by photographing the Ag—Au alloy, and FIG. 2B is a photograph obtained by photographing the nanoporous Au electrode having the first metal (Ag) removed therefrom using the nitric acid. Accordingly, a nanoporous structure can be seen.

FIG. 3 illustrate photographs obtained by photographing, for every time zone, the protruding metal electrode formed using the electron-co deposition according to the embodiment of the present disclosure. Accordingly, it can be seen that, as the deposition time is increased, a protruding region of the metal electrode, e.g., the size of the mushroom head of the mushroom structure is increased.

FIG. 4 is a conceptual view of an electrode array for an external nerve cell interface, to which the protruding metal electrode is applied according to an embodiment of the present disclosure. Referring to FIG. 4, the electrode array includes a plurality of electrodes having various sizes. According to the embodiment of the present disclosure, the size of the protruding metal electrode can be easily controlled by adjusting the time required to perform the electron-co-deposition. Accordingly, when the electrode array is manufactured, electrodes having various sizes can be easily formed without using any separate mask.

According to the present disclosure, a protruding alloy layer is formed using a mold having the structure of a micro well, and a metal included in the alloy layer is selectively removed, thereby forming a protruding nanoporous metal electrode. Thus, the protruding metal electrode can be manufactured using a micro electro mechanical system (MEMS) processing technique.

The protruding metal electrode is formed using electro-co-deposition, so that the size of the protruding metal electrode can be easily controlled by adjusting the time required to perform the electro-co-deposition. Further, as the deposition time is adjusted, the sizes of a plurality of electrodes included in one electrode array can be individually controlled without using an additional mask.

Particularly, the protruding nanoporous metal electrode is used as a neural electrode such as an electrode for extracellular neural signal detecting or an electrode for neural electrical stimulation, so that noise is reduced in detecting of neural signals, thereby improving neural signal sensitivity and increasing a charge injection limit. Further, the contact area of the protruding nanoporous metal electrode with nerve cells is increased, thereby improving signal measurement productivity.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present disclosure as set forth in the following claims.

What is claimed is:

1. A method for manufacturing a metal electrode, the method comprising:
forming a resist pattern of which upper portion has a wider width than a lower portion thereof;
forming an insulating layer for molding on the resist pattern;
removing the resist pattern, thereby forming a mold; and
forming, in the mold, a metal electrode including an alloy of a first metal and a second metal.

2. The method of claim 1, wherein the forming of the resist pattern includes:
forming a lift-off resist;
forming a negative photoresist on the lift-off resist; and
patterning the negative photoresist and the lift-off resist, thereby forming the resist pattern including a lift-off resist pattern and a negative photoresist pattern.

3. The method of claim 2, wherein, in the forming of the insulating layer for molding, the insulating layer for molding is formed to surround a sidewall of the lift-off resist pattern.

4. The method of claim 1, wherein, in the forming of the insulating layer for molding, the insulating layer for molding is coated on the resist pattern using radio frequency (RF) sputtering.

5. The method of claim 1, wherein the insulating layer for molding includes SiO₂, Si₃N₄, or Al₂O₃.

6. The method of claim 1, wherein, in the forming of the mold, the resist pattern is removed using a lift-off process.

7. The method of claim 1, wherein, in the forming of the metal electrode, the metal electrode is formed using electro-co-deposition.

8. The method of claim 1, wherein, in the forming of the mold, the mold including an empty space having the structure of a micro well is formed.

9. The method of claim 8, wherein, in the forming of the metal electrode, the metal electrode is formed in a protruding shape including a rod structure filled in the empty space and a protruding portion on the rod structure.

10. The method of claim 9, wherein, in the forming of the metal electrode, the metal electrode is formed using a deposition process, and the size of the protruding portion is increased as the time required to deposit the metal electrode is increased.

11. The method of claim 1, further comprising selectively dissolving the first metal, thereby forming a nanoporous metal electrode.

12. The method of claim 11, wherein the first metal is gold and the second metal is silver, and
the silver is selectively dissolved using a silver etchant.

13. The method of claim 11, wherein the first metal is gold and the second metal is platinum, and
the gold is selectively dissolved using a gold etchant.

14. The method of claim 1, wherein the metal electrode has a mushroom structure.

15. The method of claim 1, wherein the metal electrode is a neural electrode for extracellular neural signal detecting.

16. The method of claim 1, wherein the metal electrode is a neural electrode included in an electrode array for an external nerve cell interface.
